(19) United States
(12) Patent Application Publication
(10) Pub. No.: US 2006/0252047 A1
(43) Pub. Date: Nov. 9, 2006

(54) Device and method for analysis of samples using a combined sample treatment and sample carrier device

(76) Inventors: Simon Ekstrom, Kamnarsvagen (SE); Thomas Laurell, Skolbanksvagen (SE); Gyorgy Marko-Varga, Malmo (SE); Johan Nilsson, Bjarred (SE); Lars Wallman, Sjobo (SE)

Correspondence Address: LADAS & PARRY LLP, 224 SOUTH MICHIGAN AVENUE, SUITE 1600, CHICAGO, IL, 60604, US

(21) Appl. No.: 10/564,938

(22) PCT Filed: Jul. 14, 2004
(86) PCT No.: PCT/SE04/01134

§ 371 Date: Jul. 6, 2006

(30) Foreign Application Priority Data
Jul. 15, 2003 (SE) 03020740

(51) Int. Cl. C12Q 1/68; G01N 33/53; C12M 1/34

(52) U.S. Cl. 435/6; 435/7.1; 435/287.2

(57) ABSTRACT

The invention relates to devices and methods for chemical analysis, specifically to devices for extracting molecules, e.g. biomolecules such as peptides, and/or proteins, from a mixture of molecules in a solution and performing sample treatment on the extracted molecules before presenting them to an analysis instrument. The method for analysis of samples uses a combined sample treatment and sample carrier device. Said device comprises a plate with inlets at one side connected to respective compartments situated at respective array positions for receiving samples to be treated and analysed, each compartment being in communication with an outlet enabling fluid flow through the plate. The method comprises the steps of supplying an external container with a sample; optionally, subjecting the sample to a first treatment in the external container; transferring the sample to the combined sample treatment and sample carrier device; and subjecting the sample to a second treatment exploiting fluid flow through the device, wherein a medium is trapped in the device.

### FIELD OF INVENTION

The present invention relates to devices and methods for chemical analysis. More specifically it relates to devices for processing biological specimens. Yet more specifically it relates to devices for extracting molecules, e.g. biomolecules such as peptides, and/or proteins, from a mixture of molecules in a solution and performing sample treatment on the extracted molecules before presenting them to an analysis instrument. This analysis instrument may be optical (flourescence scanner, microscope) or a mass spectrometer. The mass spectrometry used is preferably a laser desorption/ionization (LDI) instrument, e.g. a matrix-assisted laser desorption/ionization (MALDI), surface enhanced laser desorption/ionization (SELDI) or any other form of laser desorption/ionization.

### BACKGROUND

The field of chemical and particularly biomolecular analysis is experiencing an increased demand for speed, sensitivity and economy. Mass spectrometry is a standard procedure for analysis of biomolecules (proteins, peptides, oligonucelotides) and is most efficient if the sample i.e. the biomolecules is presented to the mass spectrometer in pure form, that is without any interfering species (e.g. buffering agents, salts, acids, bases, detergents or unwanted biomolecules) present. It may also be necessary or desired to perform different enzymatic and/or chemical reactions on the sample biomolecules prior to the mass spectrometric analysis. The purification or/and affinity capture or/and enzymatic or/and chemical reaction is hence referred to as sample treatment.

Sample treatment of biomolecules are commonly performed either directly on the surface of the laser desorption/ionization plate or in a separate device/container for subsequent transfer to the laser desorption/ionization sample carrier plate. The term laser desorption/ionization plate or carrier referrers to the support used to present the sample to the mass spectrometer.

The sample biomolecules are often in a very low concentration in the solution and thus it is of great importance to keep the area of surfaces to a minimum in order to avoid unspecific adsorption of the biomolecules. One way to avoid unspecific adsorption of the biomolecules is to transfer them while bound on a medium, such as beads or/and to add the beads directly into the solution containing the analyte biomolecules directly after any sample treatment.

The problems with performing sample treatment directly on the surface of the laser desorption/ionization plate are that sample and reagents have to be transferred to the surface in a way that may lead to analyte loss or spreading on the surface. Furthermore the surface has a limited capacity for the biomolecules. The manufacture of these surfaces is often both complicated and expensive and after one use they have to be disposed.

In the case where an affinity medium on a planar surface is to capture a target biomolecule that is to be subjected to optical or MS analysis the limited capacity of the surface is especially troublesome. It is therefore of great interest to find technology/methodology that allows higher capacity binding and analysis of the captured biomolecules on a size defined area.

It is evident that any device used for the analysis of biomolecules with mass spectrometry has to be able to rapidly and in parallel handle minute amounts of samples and provide an efficient, economic and generic sample treatment process while allowing for a minimum of sample transfers. The device has to facilitate a high degree of automation, preferably using existing robotics. Finally the device should be able to present the processed sample to the mass spectrometer in a way that avoids additional transfers and provides maximum sensitivity.

In the case of protein identification with laser desorption/ionization the most commonly used protocol is that the protein is subjected to a separation step, such as gel-electrophoresis or liquid chromatography. The separated proteins are subjected to enzymatic or chemical cleavage before or after the separation, a process that may include several transfers of the sample solution, subjected to some form of sample treatment, e.g. purification and subsequently transferred onto a LDI carrier plate.

In the light of this it is obvious that there is a need for improvement in the field of devices used for sample treatment prior to mass spectrometry.

### BACKGROUND ART

Purification of peptides, proteins or oligonucelotide mixtures can be performed by passing the sample solution through a pipette tip filled with absorbent material, such as the ZipTip™, a registered trademark of Millipore Corporation. The biomolecules absorbed to the medium in the ZipTip can be eluted directly onto a MALDI target. Millipore Corporation is also marketing another product called ZipPlate™, which is a 96-well flow-through microtitre-SPE plate filled with a 0,3 μl C₁₈ resin. After purification the sample is eluted to another 96-well microtitreplate. GeLoader tips™ (Eppendorf) can be packed with capture medium to facilitate sample treatment of biomolecules prior to LDI.

Tecan markets the Tecan TecPrep 96 protein sample preparation technique that is a combination of SPE, microplate, and needle spotting technologies that offers unparalleled sample recovery with its single well processing capability and is capable of being used with numerous MALDI targets. DirectSpot® technology is especially designed for 96 well MALDI targets.

United States patent application 2002/0182649 A1 (assignee Cipergen Biosystems) discloses an apparatus and methods for protein characterization, identification, and sequencing using affinity capture laser desorption/ionization tandem mass spectrometry.

Affinity capture methods have become known for biospecific selection of certain biomolecules in connection with mass spectrometric analysis, see e.g., U.S. Pat. Nos. 6,020,208, 6,027,942, or 5,894,063 (T. W. Hutchens and T. -T. Yip). Such biospecific affinity adsorption processes can be used for purification of biomolecules.

WO0067293 to BEECHER JODY et.al., discloses a sample holder for mass spectrometry including a substrate having a surface and a film that coats the surface.

U.S. patent application 2002/0172619 A1discloses an electrospray device, a liquid chromatography device and an electrospray-liquid chromatography system. The presented electrospray device may also serve to reproducibly distribute and deposit a sample from a mother plate to daughter plate(s), such as MALDI targets, by nanoelectrospray deposition.

U.S. Pat. No. 6,124,012 to WHATMAN INC. discloses a solid phase extraction disc and apparatus for enhanced recovery and precision.

U.S. patent application 2002/0034827 A1 discloses methods for solid phase nanoextraction and desorption.

U.S. Pat. No. 6,287,872 to BRUKER DALTONIK GMBH discloses sample support plates for MALDI mass spectrometry including methods for manufacture of plates and application of sample.

U.S. patent application 2002/0045270A1 by Schurenberg, Martin et. al., discloses structured biosample support plates for mass spectroscopic analyses and procedures for manufacturing and use. The invention provides areas with affinity adsorbents adjacent to the hydrophilic anchors for purifyng biosubstances and, if wanted, for performing an affinity selection of biosubstances, whereby the finally prepared matrix sample crystals with the biosubstances for the MALDI analysis are well localised on the hydrophilic anchors.

U.S. Pat. No. 5,859,431 to FINNIGAN MAT LTD (GB) discloses a sample holder for mass spectrometer. The sample holder for use in mass spectrometry comprises a plate having a flat, which flat includes a first region having a smooth surface surrounding a second region having a rough surface. The second region defines the location for loading a sample.

U.S. patent application 2002/0155620A1 discloses a method and apparatus for desorption and ionization of analytes. The invention comprises a sample presentation apparatus for mass spectrometry. More particularly, a complex is immoblized on the sample presenting surface.

U.S. patent application 2002/0016450A1 (BBI BioSeq, Inc.) discloses a pressure-enhanced extraction and purification.

U.S. patent application 2002/0048531A1 discloses deposited thin films and their use in detection, attachment, and bio-medical applications. Applications of these thin films include desorption-ionization mass spectroscopy, electrical contacts for organic thin films and molecules, optical coupling of light energy for analysis, biological materials manipulation, chromatographic separation, head space adsorbance medium, medium for atomic molecular adsorbance or attachment, and substrates for cell attachment.

WO 00/79238 (PCT/AU00/00688) discloses an apparatus and methods for high resolution separation and analysis of compounds.

U.S. patent application 2002/0055186A1 (Oxford GlycoSciences (UK) Ltd.) presents an invention that provides a method and devices for determining the presence of proteins of interest in a sample. In practice, the method comprises submitting the sample to conditions that allow fragmentation of the proteins into target peptide fragments. The target peptide fragments are then contacted with an array of capture agents, such as antibodies, immobilized on a solid support. The capture agents recognize a target peptide fragment of a protein of interest. Binding of a target peptide fragment with an antibody is indicative of the presence of a protein of interest in the sample. The invention further provides a method for producing an array for capturing a target peptide fragment of a protein of interest, which comprises immobilizing capture agents on a solid support, wherein each capture agent specifically recognizes a sequence of a region of a target peptide fragment from a different protein of interest. The methods and arrays (devices) of the invention provide for proteomics, diagnosis, pharmacoproteomics, identification of markers of disease, and drug target discovery. The methods and arrays are particularly suitable for generating a database of information relating to protein expression.

U.S. patent application 2001/0055765A1 discloses an apparatus and methods for parallel processing of micro-volume liquid reactions.

U.S. patent application 2002/0151040A1 discloses an apparatus and methods for parallel processing of microvolume liquid reactions

U.S. patent application 2002/0160536A1 (Regnier, Fred) discloses high density sample holder for analysis of biological samples. The sample holder comprises a substrate microfabricated to define a multiplicity of microscopic islands defining sample support surfaces. At least one sump separates adjacent island surfaces and inhibits transport of samples between adjacent island surfaces.

U.S. patent application 2002/0094566A1 (Nelson, Randall W) discloses an integrated high throughput system for the mass spectrometry of biomolecules. Described is an affinity microcolumn comprising a high surface area material, which has high flow properties and a low dead volume, contained within a housing and having affinity reagents bound to the surface of the high surface area material that are either activated or activatable. The affinity reagents bound to the surface of the affinity microcolumn further comprise affinity receptors for the integration into high throughput analysis of biomolecules.

U.S. patent application 2002/0164818A1 Nelson, Randall W) discloses a mass spectrometric immunoassay analysis of specific proteins and variants present in various biological fluids. Presented herein is the construction of pipette tips (termed MSIA-Tips) containing porous solid supports that are constructed, covalently derivatized with affinity ligand, and used to extract specific proteins and their variants from various biological fluids by repeatedly flowing the fluids through the MSIA-Tips.

U.S. patent application 2001/0008615A1 (LITTLE, DANIEL P) discloses systems and methods for preparing and analysing low volume analyte elements.

U.S. patent application 2002/0137199A1 discloses microstorage, reaction and detection cells and method and apparatus for use thereof.

WO02075776 to GUSTAFSSON MAGNUS et. al., (Gyros) discloses a microfluidic system.

U.S. patent application 2002/0158195A1 (Gyros) discloses a microfluidic system. The disclosed invention relates to a method for presenting an analyte of a liquid sample as an MS-analyte to a mass spectrometer. More particularly, the method comprises the steps of applying a liquid sample containing the analyte to a sample inlet port of a microchannel structure of a microfluidic device, said structure also comprising an outlet port (MS-port) that is capable of being interfaced with a mass spectrometer, passing the analyte to the MS-port thereby transforming it to an MS-analyte, and presenting the MS-analyte to mass spectrometer via the MS-port.

U.S. patent application 2002/0000517A1 discloses a separation medium, multiple electrospray nozzle system and method. A microfabricated silicon chip with a separation material, such as in situ prepared porous polymer monoliths in its microchannels is disclosed. Control of the nozzle dimensions, applied voltages, and time provide a precise and reproducible method of sample apportionment or deposition from an array of nozzles, such as for the generation of sample plates for molecular weight determinations by matrix-assisted laser desorption/ionization time-of-flight mass spectrometry (“MALDI-TOF MS”). The capability of transferring analytes from a mother plate to daughter plates may also be utilized to make other daughter plates for other types of assays, such as proteomic screening.

U.S. patent application 2002/0000516A1 discloses a multiple electrospray device, systems and methods. A microchip-based electrospray device, system, and method of fabrication thereof are disclosed. The electrospray device includes a substrate defining a channel between an entrance orifice on an injection surface and an exit orifice on an ejection surface, a nozzle defined by a portion recessed from the ejection surface surrounding the exit orifice, and an electric field generating source for application of an electric potential to the substrate to optimize and generate an electrospray. The system is used to transfer analyte onto a MALDi target plate.

U.S. Pat. No. 6,464,866 to Moon et. al., discloses an integrated monolithic microfabricated electrospray and liquid chromatography system and method.

WO0030167 discloses a polymer-based electrospray nozzle for mass spectrometry.

U.S. patent application 2002/0094533A1 discloses an apparatus for assay, synthesis and storage, and methods of manufacture, use, and manipulation thereof. The invention features methods of making devices, or “platens”, having a high-density array of through-holes, as well as methods of cleaning and refurbishing the surfaces of the platens. The invention further features methods of making high-density arrays of chemical, biochemical, and biological compounds, having many advantages over conventional, lower-density arrays. “Still another advantage of the invention is that substances that bind to chemical probes contained in the through-hole array can easily be recovered as distinct samples for further analysis. For example, the bound contents of the well can be eluted onto a planar substrate for analysis by matrix-assisted laser desorption and ionization (MALDI) or surface-enhanced laser desorption and ionization (SELDI) mass spectrometry, or nuclear magnetic resonance (NMP) spectroscopy. Alternatively, the contents of the through-hole can be electrosprayed directly from the through-hole into a mass spectrometer. The contents of the through-hole can also be crystallised and analysed with x-ray/scintillation detection or electron diffraction techniques (e.g., to determine crystal structure). This aspect of the invention allows for sensitive detection of unlabelled analytes.”

U.S. patent application 2002/0155509A1 discloses retentate chromatography and protein chip arrays with applications in biology. “[0140] In another embodiment, the adsorbent is attached to a first substrate to provide a solid phase, such as a polymeric or glass bead, which is subsequently positioned on a second substrate which functions as the means for presenting the sample to the desorbing energy of the desorption detector. For example, the second substrate can be in the form a plate having a series of wells at predetermined addressable locations. The wells can function as containers for a first substrate derivatized with the adsorbent, e.g., polymeric beads derivatized with the adsorbent. One advantage of this embodiment is that the analyte can be adsorbed to the first substrate in one physical context, and transferred to the sample presenting substrate for analysis by desorption spectrometry.”

WO0138865 discloses an apparatus and method for trapping bead-based reagents within microfluidic analysis systems.

U.S. Pat. No. 6,432,290 discloses an apparatus and method for trapping bead-based reagents within microfluidic analysis systems.

U.S. patent application 2002/0168644A1 (Aebersold, Rudolf H) discloses methods for isolation and labelling of sample molecules. The invention provides methods for labelling a molecule by contacting a sample molecule with a solid support coupled to a chemical group comprising a cleavable functional group, one or more functional groups, and a reactive group for the sample molecule, under conditions allowing the sample molecule to covalently bind to the reactive group; and cleaving the cleavable functional group, thereby releasing the sample molecule comprising the one or more functional groups, which can be a tag.

U.S. patent application 2003/0032046A1 discloses peelable and resealable devices for biochemical assays.

U.S. patent application 2003/0032013A1 discloses high capacity assay platforms. A high capacity assay platform capable of binding target molecules includes a substrate and a polymer matrix attached to the substrate. The substrate may be a MALDI plate.

U.S. patent application 2002/0182114A1 discloses a device for processing samples, use of the device, and method for producing the device. “The invention relates to a device (1) for processing samples (2), comprising a body (3) with a collecting chamber (4), which can be connected with a pump (5) for aspirating, or dispensing fluids and that acts on this collecting chamber, a separating chamber (6) adjoining the collecting chamber (4) for the solid phase extraction and elution of organic, or inorganic particles (7) separated from these samples (2), and an opening (8) for releasing these particles (7). The device in accordance with the invention relates to individual pipette tips, as well as to SPE microplates, and is distinguished in that the device (1) comprises a capillary (9), which is connected with the collecting chamber (4), or with the body (8), and has a packing (10) for the solid phase extraction of organic, or inorganic particles (7) separated from these samples (2) and is used as a separating chamber (6). In accordance with the invention, the package (10) can be adapted to the chemical-physical nature of the organic, or inorganic particles (7) to be extracted, as well as to a defined minimum volume.”

U.S. Pat. No. 5,705,813 discloses an integrated liquid sample handling system for matrix-assisted laser-desorption/ionization time-of-flight mass spectroscopy (MALDI-TOF MS).

### SCIENTIFIC PUBLICATIONS

Many scientific publications have covered different aspects of sample treatment prior to LDI. To date none of these publications has presented a generic device that addresses the need for rapid automated and parallel handling of minute sample amounts in an efficient and economic way that also allows for generic sample treatment processes while allowing for a minimum of sample transfers and the ability to present the processed sample to the mass spectrometer in a way that avoids additional transfers and provides maximum sensitivity.

A number of publications have presented methodology that enables efficient analysis of biomolecule, though. Many methodologies that rely on the use of particles, beads, membranes, Empore discs or similar for sample treatment of biomolecules prior to LDI can be accommodated and/or improved on the present invention (combined sample support and carrier device).

### SUMMARY OF THE INVENTION

In a first aspect the present invention provides a device for combined sample treatment and sample carrying, comprising a plate with inlets at one side connected to respective compartments situated at respective array positions for receiving samples to be treated and analysed, characterised in that each compartment is in communication with an outlet enabling fluid flow through the plate

In a second aspect the present invention provides a method for analysis of samples using a combined sample treatment and sample carrier device comprising a plate with inlets at one side connected to respective compartments situated at respective array positions for receiving samples to be treated and analysed, each compartment being in communication with an outlet enabling fluid flow through the plate comprising the steps of:
supplying an external container with a sample;
optionally, subjecting the sample to a first treatment in the external container;
transferring the sample to the combined sample treatment and sample carrier device;
subjecting the sample to a second treatment exploiting fluid flow through the device, wherein
a medium is trapped in the device.

The invention is defined in the attached claims.

### BRIEF DESCRIPTION OF THE DRAWINGS

Embodiments of the invention is disclosed in the following description and described with the aid of the following figures in which:

FIG. 1a is a perspective view of one embodiment of the combined sample support and carrier plate according to the invention;

FIG. 1b is a cross section view of such a plate;

FIGS. 1c to 1n are views of various shapes of the compartment in a plate;

FIG. 2a is a perspective view of another embodiment of the combined sample support and carrier plate according to the invention;

FIGS. 2b to 2i are views of various shapes of restrictions in channels in the embodiment of FIG. 2a;

FIG. 3a is a perspective view of another embodiment of the combined sample support and carrier plate according to the invention;

FIGS. 3b to 3d are various views of the compartment in the embodiment of FIG. 2a;

FIG. 4a is a perspective view of another embodiment of the combined sample support and carrier plate according to the invention;

FIGS. 4b to 4d are various views of alternative compartments in the embodiment of FIG. 4a;

FIG. 5a is a perspective view of another embodiment of the combined sample support and carrier plate according to the invention;

FIG. 5b is a side view of two compartments in the embodiment of FIG. 5a;

FIGS. 6a to 6w are various views of various embodiments of the structure at outlets of the plate according to the invention;

FIGS. 7a and 7b are side views of various forms of media applied in compartments;

FIGS. 8a to 8d are various schematic views of methods for applying media and analytes;

FIGS. 9a to 9d are schematic views of solid-phase purification of analytes with the plate according to the invention;

FIGS. 10a to 10d are schematic views of different detection principles for analysis with the plate according to the invention;

FIG. 11a is a perspective view of another embodiment of the combined sample support and carrier plate mating with another plate;

FIG. 11b is a side view of the two plates in FIG. 11a;

FIG. 12 is a perspective view of the combined sample support and carrier plate according to the invention connected to a sample basin;

FIG. 13a is a perspective view of another embodiment of the combined sample support and carrier plate in which three plates are stacked together;

FIG. 13b is a side view of the three plates in FIG. 13a;

FIG. 14a is a perspective view of another embodiment of the combined sample support and carrier plate according to the invention arranged for electrospray;

FIG. 14b is a side view of the compartment and nozzle arrangement of the plate in FIG. 14a;

FIGS. 14c to 14e are perspective views of nozzle embodiments;

FIG. 15a is a perspective view of yet another embodiment of the combined sample support and carrier plate according to the invention arranged for electrospray;

FIG. 15b to 15d are side views of the various compartment and nozzle arrangements of the plate in FIG. 15a; and

FIGS. 15e to 15g are perspective views of nozzle embodiments.

### DESCRIPTION OF PREFERRED EMBODIMENTS

### Introduction

The present invention provides a combined sample support and carrier device. The device allows for selectively retrieving and concentrating biomolecules from solutions and subsequent presentation of the treated biomolecules to LDI MS and/or MSⁿ for final analysis.

The combined sample treatment and carrier plate has a number (2-10000) of array positions having an inlet to a compartment that can be filled with capture medium that gets entrapped in the compartment. Each array position also has an outlet with a separate analysis zone where the individual samples can be analysed.

The capture medium can be of any known type such as non-selective affinity adsorbents or affinity capture/adsorption medium. The medium can be particles or beads of e.g. silica, glass, metal, or polymeric materials. In the case where the medium consists of beads or particles the retaining of the medium can be accomplished by designing the outlet hole or holes in such a way that the size of an individual particle or bead is bigger than the size of the outlet hole/holes. It is also possible to use beads or particles having a size that is less (up to 5 times smaller) than that of the outlet hole/holes and rely on the “keystone” effect for retaining the medium in the medium compartment of the combined sample treatment and carrier plate. The medium may also be supplied to the combined sample treatment and carrier plate by in-chip (in-situ) polymerisation of medium e.g. any type of porous polymer monolith.

Yet another way to transfer the medium into the combined sample treatment and carrier plate is to place pieces of capture medium, such as e.g. pieces of membranes, Empore Disks (3M, Minneapolis, Minn.) or similar, in the medium compartments of the combined sample treatment and carrier plate.

The combined sample treatment and carrier plate allows for efficient washing of the captured biomolecules to ensure that no undesired species are transferred to the analysis zone in the elution step. This provides increased sensitivity and specificity during the analysis.

The methodology where the biomolecules are put in contact with the medium without any sample transfers and as soon as possible after any sample treatment (e.g. the media are added directly into a microtitreplate after a protein digestion or a separation of biomolecules is eluted directly onto the medium) of the biomolecules provides a significant advantage since it is well know that low concentrations of biomolecules quite quickly becomes unspecifically adsorbed to surfaces (such as surfaces of sample containers). The rapid capture of the biomolecules onto the medium ensures that they can be recovered for analysis later rather than disappear due to unspecific adsorption. This means that more of the desired biomolecules are available for the final analysis, i.e. a better analytical result is obtained. Furthermore when the biomolecules get exposed to the capture medium and biomolecules in the sample solution bind to the medium the concentration of biomolecules in solution becomes lower. This can be used to advantage in situations where the desired biomolecules are continuously created by another sample treatment process or the desired biomolecules are leaking out into the solution over a time frame, e.g. from a gel-plug.

The use of microfabrication to realize the combined sample treatment and carrier device provides several beneficial aspects. The miniaturized combined sample treatment and carrier device requires small sample and reagent volumes, and allows for a high-density of array positions. Another important aspect is the short path length in the combined sample treatment and carrier device, where the analytes are loaded directly on the media and either analysed on the media or on the outlet analysis zone, allowing for minimal unspecific adsorption of the analytes on surfaces in the device. This is a significant improvement compared to devises, e.g. microfluidic systems such as that presented in WO0275776, where the sample analytes are introduced and eluted through a channel having a considerably larger surface area. Although the combined sample treatment and carrier device also can be realized through the use of buried channels the preferred embodiments are those with short path lengths, since devices using buried channels are more complicated and thus also more expensive to manufacture.

The basic idea is that the device can be used in a number of generic applications. The user chooses a capture medium or several different (complementary) capture medium types, depending on the application, to fill the combined sample treatment and carrier plate with. The desired biomolecules can be captured on the medium off-line in an external container, e.g. a microtitreplate, before loading into separate array positions on the combined sample treatment and carrier plate, or the medium can be loaded into the individual array positions first and the sample solution containing the desired biomolecules are then passed through the medium for capture. Undesired components that may still be present or bound to the medium are then washed away, leaving the desired biomolecules on the medium. The desired biomolecules are now captured on the medium where they can be subjected to additional sample treatment steps. These steps can be enzymatic treatments or/and chemical treatments, e.g. made to promote analysis or/and obtain additional information about the captured biomolecules or initial analysis of the biomolecules can be made. After all desired sample treatments steps are completed the biomolecules are eluted, by an eluant fluid that desorbs the biomolecules causing them to solve, onto the analysis zone on the combined sample treatment and carrier plate. The biomolecules can now be subjected to even further sample treatment steps on the analysis zone. If no additional sample treatment is desired the plate can be subjected to analysis by LDI MS or the plate can be stored for later analysis. The combined sample treatment and carrier plate is manufactured in a way that allows it to fit directly into the LDI MS instrument or in such a way that it fits into a substrate that serves as holder, allowing insertion of the combined sample treatment and carrier plate into the LDI MS instrument.

Some embodiments of the present invention can also be used for presenting the sample biomolecules to an electrospray MS instrument (ESI). In these embodiments one or several electrodes are incorporated in the combined sample treatment and sample carrier plate. These electrodes are used generate the electrospray/nanospray or the can be used to promote analyte transport to or from the medium.

An advantage is that as soon as the desired biomolecules are captured on the medium in the combined sample treatment and carrier plate, the subsequent steps can be performed at a later time, i.e., the combined sample treatment and carrier plate can be stowed away until analysis is to be performed.

The elution of the captured biomolecules onto the analysis zone of the combined sample treatment and carrier plate can be made with an eluting solution that contains the matrix necessary for MALDI or any other component necessary for LDI. This matrix or/and component (chemicals, metal powder, semiconductor powder) to enable/promote LDI can also be applied to the analysis zone before elution of the captured biomolecules or after the elution of the captured biomolecules onto the analysis zone, by e.g. dispensing, spraying, vapour deposition, spinning or chemical binding

The combined sample treatment and carrier plate is easily interfaced to ordinary pipetting robots for loading of beads, sample, wash and elution solutions. The sample treatment using the combined sample treatment and carrier plate minimises sample transfers and thus also unspecific adsorption of the analyte biomolecules on surfaces, e.g. pipette tips, sample containers, connective tubing.

The combined sample treatment and carrier plate finally presents the analyte biomolecules on the analysis zone of the combined sample treatment and carrier plate to the LDI MS instrument. This means that no transfers of the analyte biomolecules to a separate LDI MS plate has to be made, avoiding adsorption losses of the analyte and also the need for additional steps manually or made by robotics to perform this transfer.

By placing a second plate of through-hole wells (matching the inlets of the combined sample treatment and carrier plate) on top of the combined sample treatment and carrier plate that can hold a gel plug excised from a 2-DE separation of proteins, the whole process of in-gel digestion, peptide capture (concentration/purification of the peptides), elution of captured peptides onto the analysis zone of the combined sample treatment and carrier plate and LDI MS analysis can be made without any sample transfers outside of the combined sample treatment and carrier plate, i.e. all the steps after the gel-plug has been put in its well are performed in the same single robotic station.

In order to facilitate the forming of small spots during the elution of the analyte biomolecules onto the analysis zone of the combined sample treatment and carrier plate, the combined sample treatment and carrier plate can be treated with a hydrophobic agent and/or the analysis zone can be structured in a way that promotes the forming of a small high analyte density area on the analysis zone. The structuring of the combined sample treatment and carrier plate surface can also serve to enable docking to other plates (e.g. a second combined sample treatment and carrier plate) or it can enable loading of larger volumes sample.

The use of vacuum applied to the backside (i.e. the side with the analysis zones) to achieve fluid transport provides a means to control several important parameters of the LDI sample preparation process. These parameters include spot size, the crystal growth and size.

The size of the combined sample treatment and sample carrier plate can be of any dimension but preferably it has the same dimension as the standard target plate of the analysis instrument that the combined sample treatment and sample carrier plate is supposed to be analysed with.

### Description with Reference to the Drawings

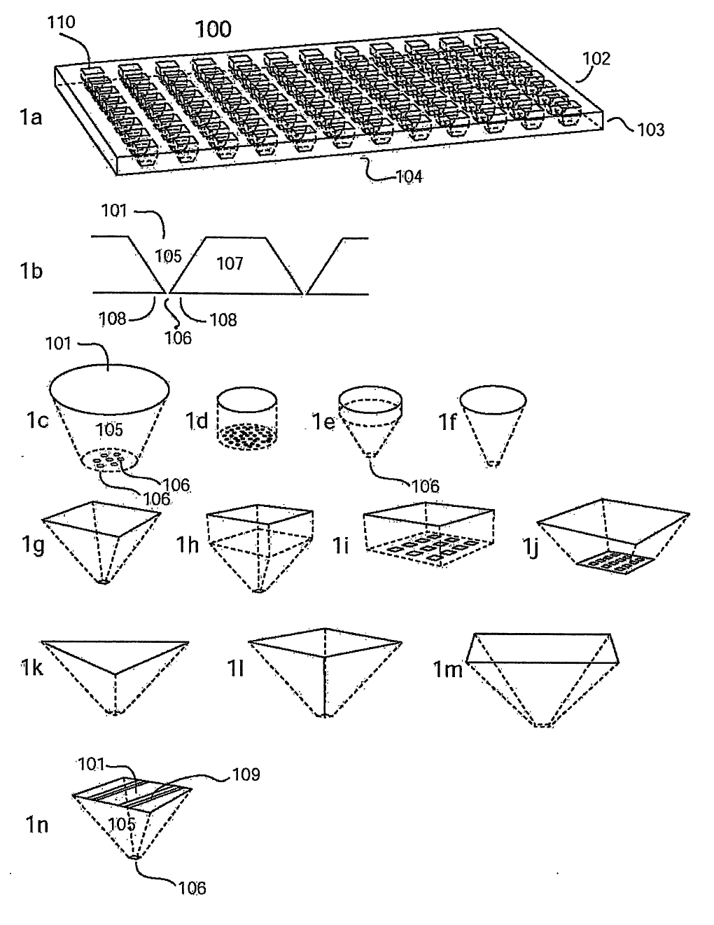

FIG. 1 shows an example of the combined sample treatment and sample carrier plate and some possible designs of the inlet (101), the medium compartment (105), the outlet (106) and the analysis zone (108)

FIG. 1a shows an embodiment of the combined sample treatment and sample carrier plate (100) having 96 individual array positions (110) . Each side (102) and (104) can typically be between 4-20 cm and the thickness (103) of the combined sample treatment and sample carrier plate (100) can typically be between 1 μm to 2 cm, but preferably between 200 μm-4 mm.

Each array position (110) has a defined geometry and this can be of the same type on the entire combined sample treatment and sample carrier plate, or the combined sample treatment and sample carrier plate can have a multitude of array positions of different shapes.

FIG. 1b depicts a side view of two array positions in one of the preferred array geometries. The inlet 101 can have a side/diameter between 500 nm-2 cm, but preferably has a side/diameter between 100-500 μm, and the outlet 106 can typically have a side/diameter of 1 nm-2 cm, but preferably has a side/diameter between 1-50 μm. The analysis zone (108) is the area surrounding the outlet (106). The inlet (101) and outlet (106) defines a compartment (105), having a volume defined by the geometry. The compartment (105) can be loaded with any type of medium having any desired function.

The inlet (101) and outlet (106) of an array position (110) can have any known geometry, e.g. square, rectangular, circular, oval, triangular, rhombic, octahedral, etc. The outlet and inlet can have the same geometry but it is also possible to have different geometries on the outlet and the inlet, e.g. a circular inlet and a square outlet. The array position (110) can also be of any type having multiple outlets, see e.g. FIGS. 1c, 1d, 1i and 1j.

The geometry of an array position (110) can have any known form e.g. conical as in FIGS. 1c, 1f or cylindrical as in FIG. 1d, or a combination thereof, FIG. 1e. FIGS. 1g-1j shows some examples of square and pyramidal geometries. FIG. 1k is an example of a triangular geometry, FIG. 1l rhombic and FIG. 1m a rectangular geometry.

It is also possible to include structures to the inlet (101) and/or outlet (106) and/or the compartment (105). FIG. 1n depicts such a structured design, where bars (109) have been included in the inlet (101) to keep large matter from entering the compartment (105) while allowing for the medium to fill the compartment (105).

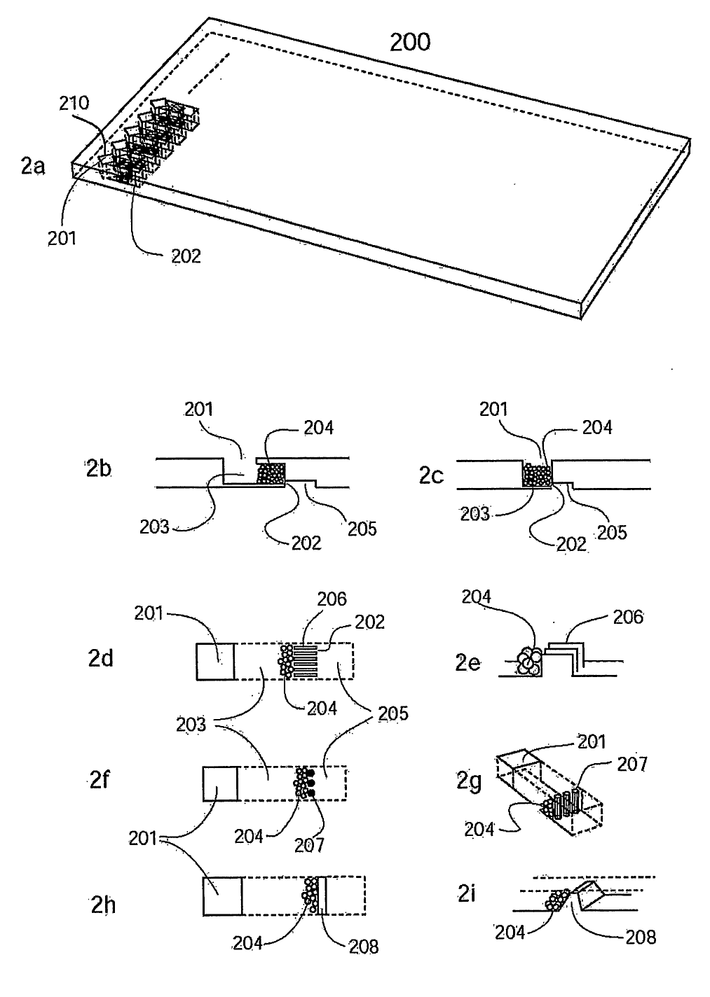

FIG. 2a depicts another embodiment of the combined sample treatment and sample carrier plate (200) having a multitude of individual array positions (210). Each array position (210) has an inlet (201) and an outlet (202) that defines a compartment (203). The compartment can be filled with medium (204).

FIG. 2b shows a side view of an array position embodiment. The array position has an inlet (201), an outlet (202) and a compartment (203) that can be filled with medium (204). In this embodiment the inlet and outlet does not define the volume of the compartment. Instead the compartment extends as a channel in directed in the same plane as the plate (horizontally) in the material comprising the combined sample treatment and sample carrier plate. This allows for a compartment of a larger volume as compared to a compartment defined solely by the inlet and outlet. The outlet is a narrow opening that allows for the retaining of the medium (204). The outlet leads out into a structured analysis zone (205). This structuring can serve to define the size of the analysis zone.

FIG. 2c is another embodiment of the design depicted in FIG. 2b, but here with an array position with an inlet (201) and outlet (202) that defines the compartment (203) volume.

FIG. 2d top view and FIG. 2e side view show an array position design that utilises a number of walls (206) that form a grid to retain the medium.

FIG. 2f top view and FIG. 2g side view show an array position design that utilises a number of pillars (207) that form a grid to retain the medium.

FIG. 2h top view and FIG. 2i side view shows an array position design that utilises a heel (208) that provides a restriction allowing for the capture and retention of the medium.

The designs in FIGS. 2d-2i have in common that the compartment (203) volume can be expanded by letting the compartment extend horizontally in the material comprising the combined sample treatment and sample carrier plate. It should also be noted that by moving the restriction structure (206, 207, 208) to the rim of the inlet a design like that presented in FIG. 2c, that has a compartment (203) volume defined by the inlet and outlet, can be realized.

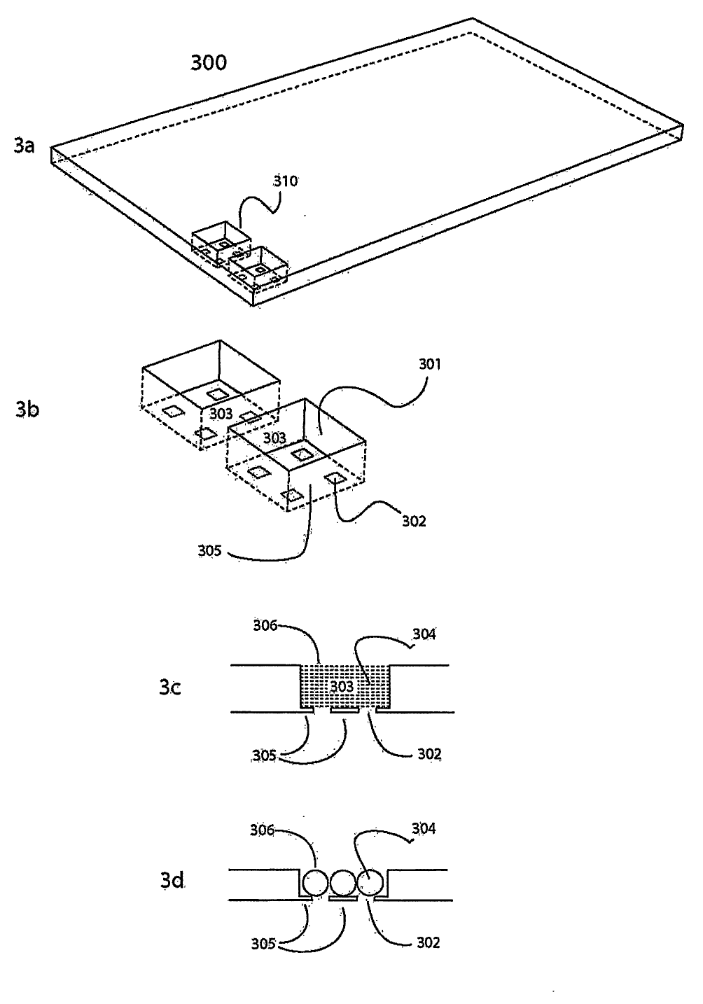

FIG. 3a depicts an embodiment of the combined sample treatment and sample carrier plate (300) where each individual array position (310) has a very shallow medium capture compartment (303). FIG. 3b shows a closer view of two array positions. The volume of the compartment (303) is defined by the inlet (301) and outlet (302). This enables easy and direct analysis of analytes captured on the medium (304), i.e. analysis without elution of the captured analytes onto the analysis zone. The compartment (303) can have any depth but in the embodiment (300) it is preferred that medium (304) is level with the inlet (301) as shown in FIGS. 3c and 3d, since the inlet (301) in this embodiment (300) also serves as analysis zone. More specifically the medium (304) surface is the analysis zone (306). It should be noted that the surface surrounding the outlet (302) also could serve as analysis zone (305).

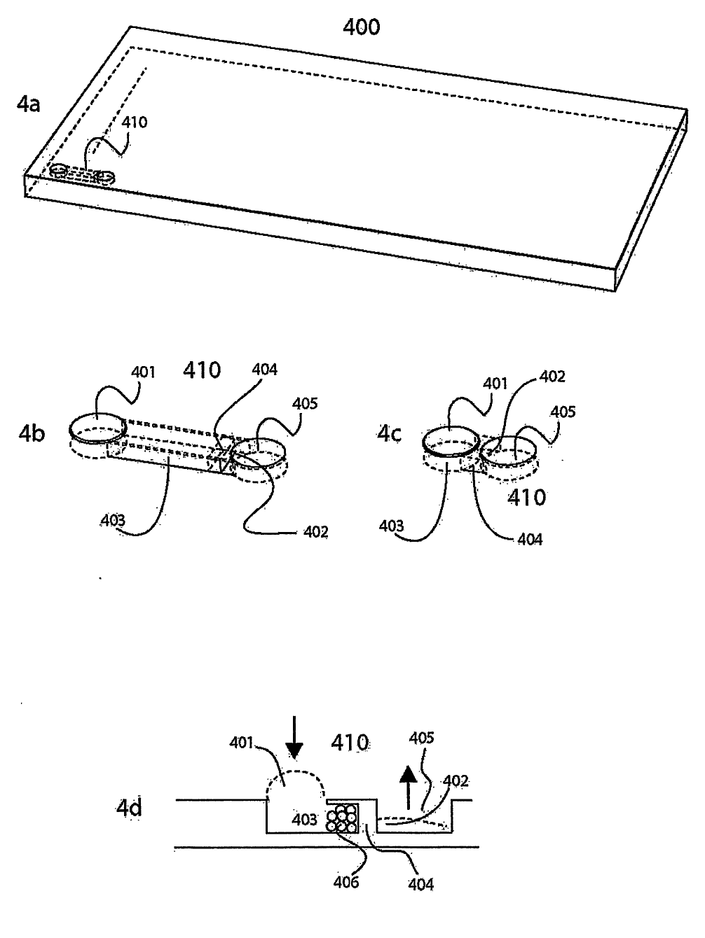

FIG. 4a depicts an alternative embodiment of the combined sample treatment and sample carrier plate (400) having a multitude of array positions (410) that has the inlet (401), the outlet (402) and the analysis zone (405) on the same side of the combined sample treatment and sample carrier plate. FIG. 4b shows a design where the compartment (403) volume can be expanded by letting the compartment extend as a channel directed in the same plane as the plate (horizontally) in the material comprising the combined sample treatment and sample carrier plate. It should also be noted that by moving the restriction structure (404) to the rim of the inlet (401) and the rim of the outlet (402) a design like that presented in FIG. 4c, that has a compartment (203) volume defined by the inlet and outlet, can be realized. The restriction structure (404) can be of any known type, such as pillars, walls, heel or openings. FIG. 4d shows a side view of an array position (410) like that in FIG. 4b and the arrows indicate the direction of flow through the compartment (403) filled with medium (406).

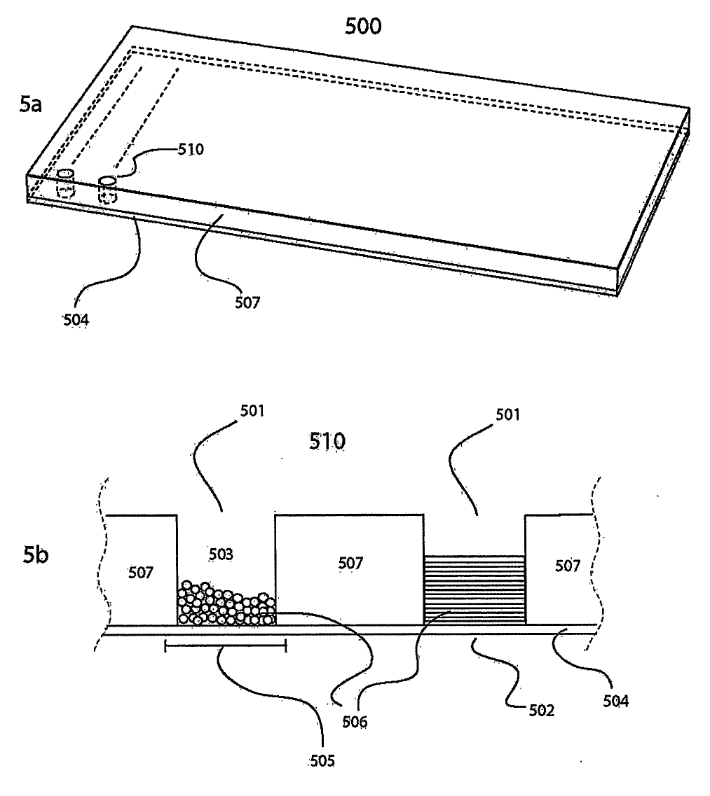

FIG. 5a depicts yet another alternative embodiment of the combined sample treatment and sample carrier plate (500) having a multitude of array positions (510). FIG. 5b shows a side view of two array positions (510), each filled with a different medium (506). The array positions (510) in the substrate (507) can have any shape and geometry. The difference here is that the restriction (504) that serves to retain the medium (506) is a thin permeable membrane (504). Any liquid delivered to the inlet (501) passes through the compartment (503) and the membrane (504) that is permeable i.e. has passages/outlets (502). The analysis zone (505) is the area where the analytes end up after elution from the medium (506).

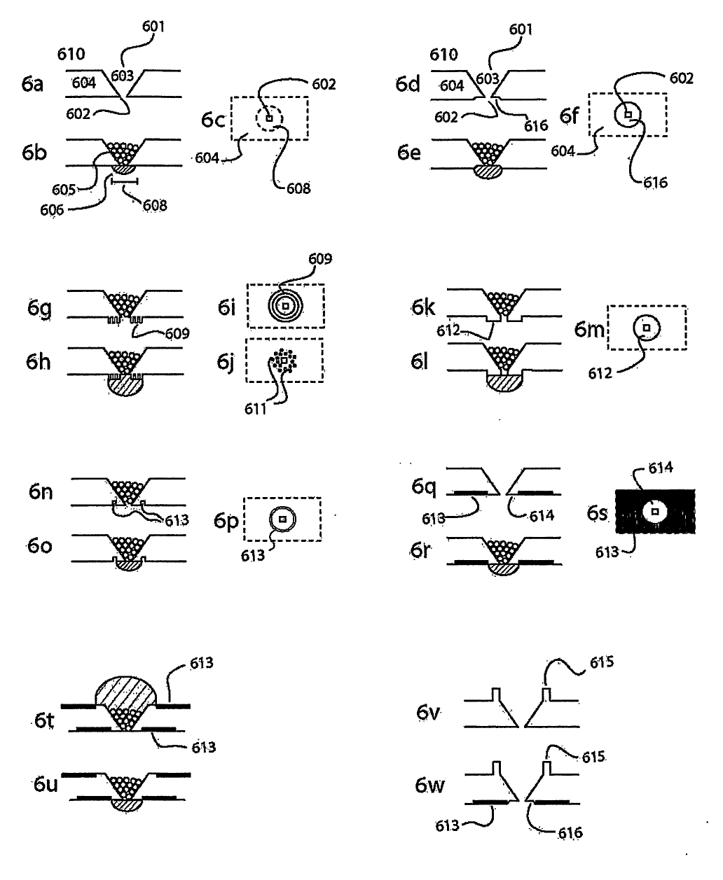

As is shown in FIG. 6, the array positions of the combined sample treatment and sample carrier plate can have any known type of additional structuring on the inlet and/or medium compartment and/or outlet (analysis zone) and the area surrounding these. The structuring can serve to allow for different desired properties, e.g. allow loading of larger volumes, higher loading capacity of medium, enhance or facilitate the analysis, or facilitate the creation of a small area defined sample spot during the elution of the captured analytes onto the analysis zone.

FIG. 6a shows a side view of a non structured array position (610) in the plate material (604) comprising the combined sample treatment and sample carrier plate with an inlet (601) and an outlet (602) that defines a compartment (603). FIG. 6b shows a side view of the resulting droplet (606) on the analysis zone (608), surrounding the outlet (602), directly after elution of desired analytes from the medium (605). FIG. 6c shows a view of the bottom of the combined sample treatment and sample carrier plate, with the outlet (602) and the analysis zone (608). FIG. 6d shows a side view of a structured array position (610) in the plate material (604) comprising the combined sample treatment and sample carrier plate with an inlet (601) and an outlet (602) that defines a compartment (603). The analysis zone is defined by the structuring of a well (616) at the outlet (602). FIG. 6e shows a side view of the confinement of the eluted liquid due to the well (616). FIG. 6f shows a view of the bottom of the combined sample treatment and sample carrier plate, with the outlet (602) and the well/analysis zone (616). FIG. 6g shows a side view of another embodiment where the structured analysis zone is a set of concentric circular walls (609) surrounding the outlet and FIG. 6h shows the confinement of the elution liquid. FIG. 6i is a bottom view of FIG. 6g with the circular walls (609). FIG. 6j shows another possible structuring in the form of pillars (611). FIG. 6k shows how the analysis zone can be defined with a chimney like structure (612). FIG. 6L shows the confinement of the elution liquid and FIG. 6m a bottom view of the structuring. In figure FIGS. 6n-p a trench (617) structuring is used to confine the elution liquid on the analysis zone.

FIGS. 6q-s shows how a patterning/structuring (613, 614) can be used to confine the elution liquid on the analysis zone. The patterning/structuring can be added to the combined sample treatment and sample carrier plate by any known means and be of any known type, such as hydrophobic (614) and hydrophilic (613) or nanoporous (614) and planar (613). This patterning/structuring can of course be applied to the area surrounding the inlet and the outlet as shown in FIGS. 6t-u. FIG. 6v shows a structuring (615) applied to the inlet. The pattering and structuring can thus be applied to the areas surrounding both the inlet and outlet and/or the inlet, the compartment and/or the outlet in any combination, FIG. 6w, in order to facilitate specific attributes of the combined sample treatment and sample carrier plate.

It is also possible to make any of the sides of the device hydrophobic or make the entire surface of the device hydrophobic.

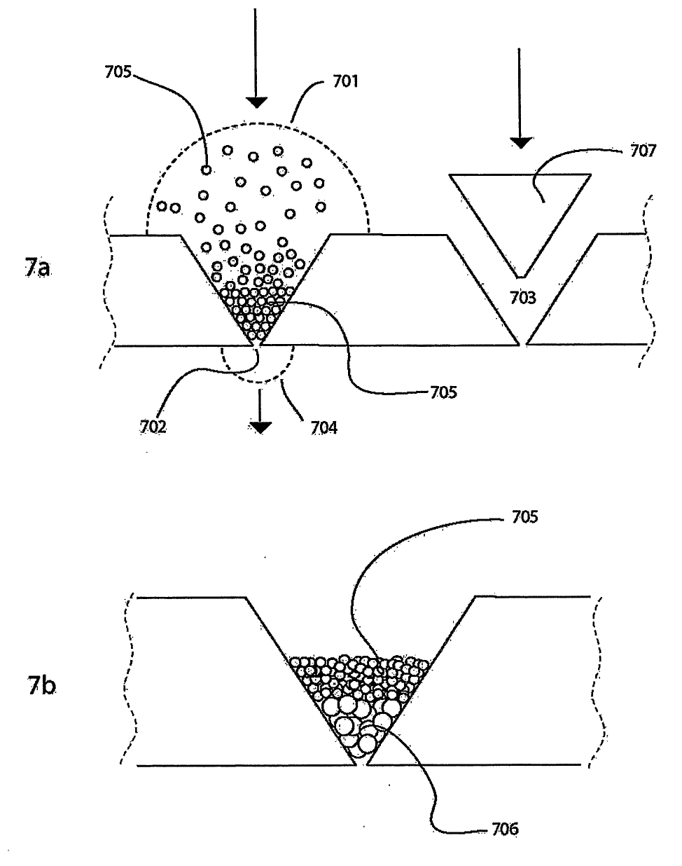

As is shown in FIG. 7, media (705-707) of any known type can be loaded into the compartment (703) by any known means, e.g. in-situ polymerisation, beads in slurry or pieces of medium such as Empore disks™ (3M).

FIG. 7a shows an example of loading two different medium types into the medium compartment (703) of individual array positions on the combined sample treatment and sample carrier plate. The medium can be loaded in the form of a bead suspension (705) that is applied to the inlet (701) and a vacuum can be applied under the combined sample treatment and sample carrier plate to pack the medium into the medium compartment (703). The medium (705) will be retained in the compartment (703) while the solvent (704) used in the bead suspension will pass through the outlet (702). Another way of loading the medium is to place medium pieces (707) that fits into the compartment (703).

FIG. 7b shows how a single array position on the combined sample treatment and sample carrier plate can be filled with medium that has different functions. The different medium types can be used in order to obtain desired physical features, e.g. large beads (706) can be used to keep small beads (705) in the compartment or in order to allow for specific and multidimensional sample treatment steps.

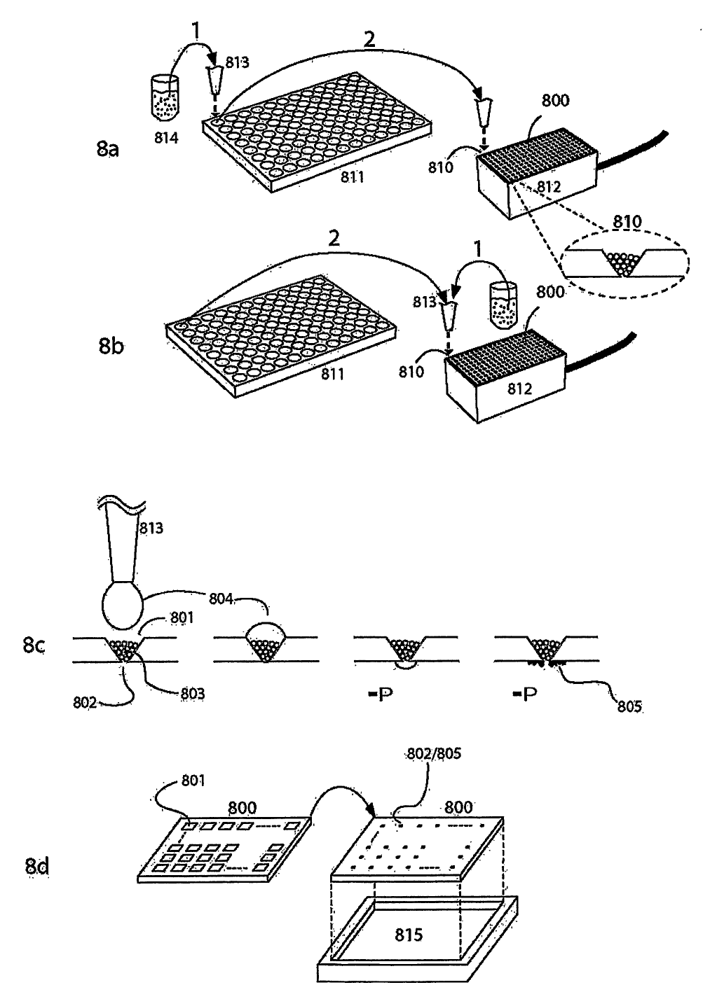

FIG. 8 shows some different strategies for use of the combined sample treatment and sample carrier plate (800).

In FIG. 8a the medium is first (1) added in the form of a bead slurry (814) to the analytes in an external container (811), e.g. a microtitreplate by e.g. a pipetting robot (813). The medium captures the desired analytes in the array position of the external container (811) and is subsequently transferred (2) to corresponding array position (810) on the combined sample treatment and sample carrier plate (800). The combined sample treatment and sample carrier plate is placed in a fixture (812) that enables loading of the medium and other sample treatment processes by e.g. applying a vacuum to the bottom of the combined sample treatment and sample carrier plate.

In FIG. 8b the medium is first (1) added in the form of a bead slurry (814) to the compartments of the array positions (810) in the combined sample treatment and sample carrier plate (800) by a pipetting robot (813).

The sample solution containing the desired analytes is then transferred (2) from an external container (811) to the corresponding array position (810) on the combined sample treatment and sample carrier plate (800). The desired analytes are then allowed to bind to the medium where additional sample treatment processes can be performed before the analytes are eluted onto the analysis zone.

FIG. 8c shows an example of the elution procedure. A pipetting robot (813) delivers an appropriate amount of elution liquid (804) to the inlet (801) of an array position (810). A vacuum applied under the combined sample treatment and sample carrier plate (800) ensures that the elution liquid is transferred through the compartment and outlet (802). Thus, the elution liquid displaces the desired analytes captured on the medium (803) in the compartment and the analytes end up on the analysis zone (805). The (−P ) denotes a vacuum applied under the combined sample treatment and sample carrier plate.

As is shown in FIG. 8d, after elution of analytes to the analysis zone the combined sample treatment and sample carrier plate is turned up side down so that the outlet (802)/analysis zone (805) faces upwards. The combined sample treatment and sample carrier plate (800) can now be inserted directly in a suitable instrument that allows analysis of the combined sample treatment and sample carrier plate, or, if necessary, first be placed in a fixture (815) to be adapted to the instrument.

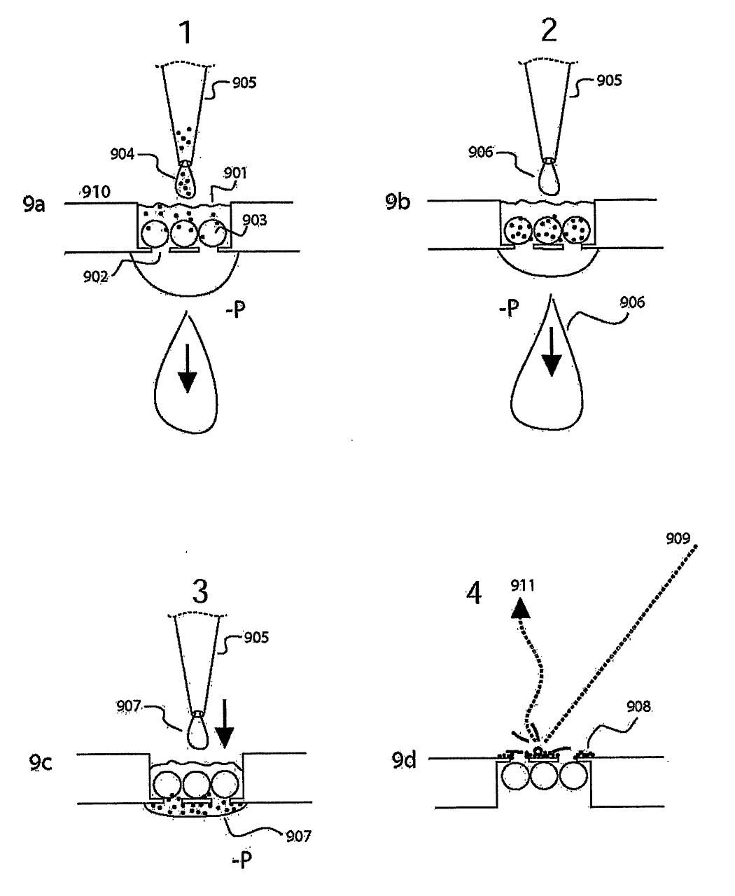

FIG. 9 shows simple schematic example of solid-phase purification of analytes with the combined sample treatment and sample carrier plate. The (−P ) denotes a vacuum applied under the combined sample treatment and sample carrier plate.

In FIG. 9a, first (1) the sample (904) is delivered to the inlet (901) of an array position (910) by any known method (905). The desired analytes binds to the medium (903) in the medium compartment, while most of the undesired components passes through the outlet (902).

FIG. 9b shows the second step (2) where remaining undesired components are washed away by a washing solution (906).

FIG. 9c shows the third step (3) where the purified and concentrated analytes are eluted from the medium onto the analysis zone (908) with an elution liquid (907).

FIG. 9d shows the final LDI MS analysis step (4) where the combined sample treatment and sample carrier plate is turned upside down and inserted into the analysis instrument and each analysis zone (908) is interrogated with a laser (909) causing ionisation (911) of the analytes.

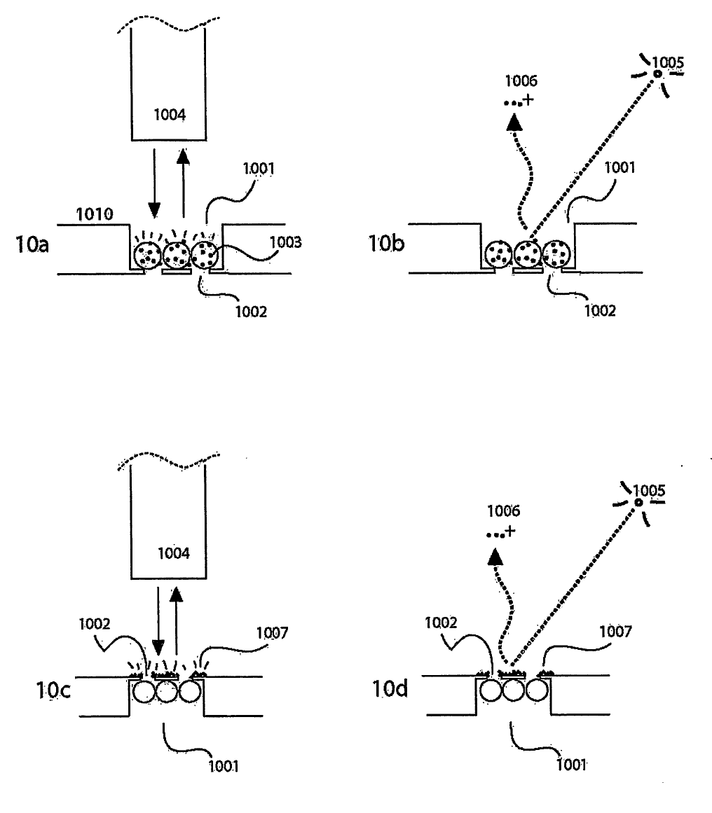

FIG. 10 shows an example of different detection principles that can be used for analysis of captured analytes on the combined sample treatment and sample carrier plate.

The analytes in an array position (1010) can be analysed by optical, FIG. 10a, or laser, FIG. 10b (1005), desorption/ionization (1006) while still bound to the medium (1003), i.e analysis from the inlet (1001), with the outlet (1002) facing down.

Alternatively the analytes are eluted onto the analysis zone (1007) surrounding the outlet (1002), i.e analysis with the outlet (1002) facing upwards to the analysis instrument. The analysis instrument can be optical e.g. fluorescence analysis, FIG. 10c, or laser as in FIG. 10d (1005), desorption/ionization (1006).

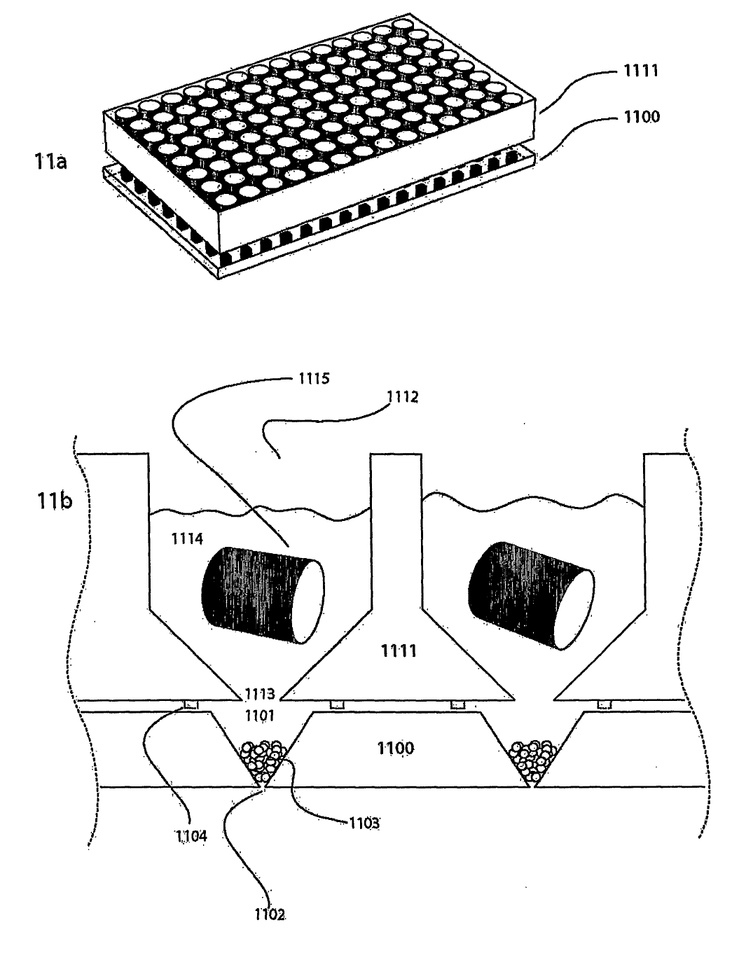

As is shown in FIG. 11, in order to facilitate some processes, e.g. minimise sample-transfers and simplify automation of the sample treatment protocols, the combined sample treatment and sample carrier plate (1100) can be mated to an additional sample treatment plate (1111) that also is of flow-through type. This additional sample treatment plate (1111) can be of any geometry and function, while manufactured to fit on top or under the combined sample treatment and sample carrier plate (1100).

FIG. 11b shows an example (side view) of mating an additional sample treatment plate (1111), allowing for in-gel digestion processing, with the combined sample treatment and sample carrier plate (1100). The additional sample treatment plate (1111) has an inlet (1112) and an outlet (1113) defining a compartment (1114) that can hold a gel-plug (1115). The outlet (1113) has a size that allows the retaining of the gel-plug (1115) and selective retention of the liquid in the compartment (1114), e.g. the liquid only passes through the outlet (1113) to the inlet (1101), the medium (1103) and/or the outlet (1102) of the combined sample treatment and sample carrier plate (1100) when a vacuum is applied under the combined sample treatment and sample carrier plate (1100). In order to improve the mating of this additional sample treatment plate (1111) to the combined sample treatment and sample carrier plate (1100) a structure (1104), e.g. an O-ring, that facilitates this mating can be included on both or either of the plates (1111 or 1100).

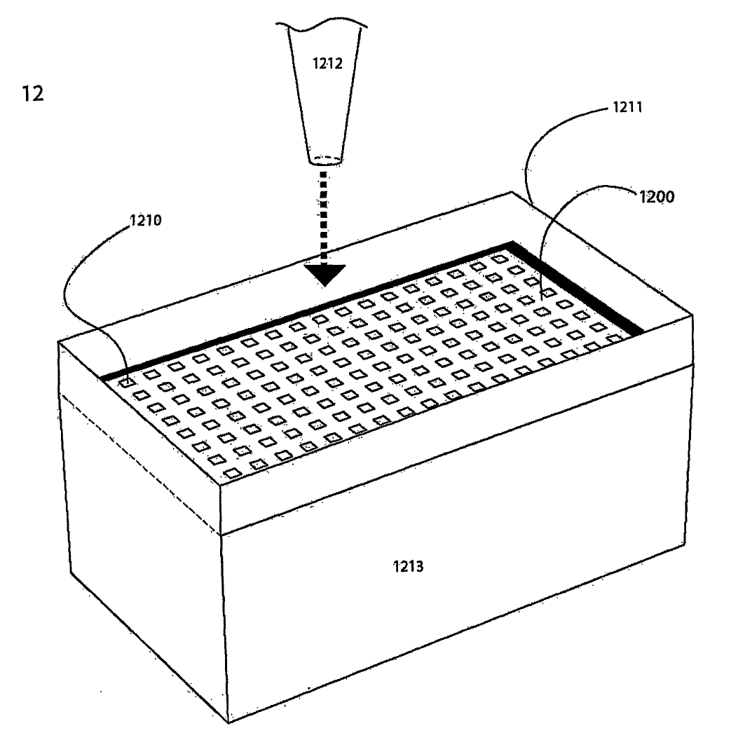

FIG. 12 shows an example of how the combined sample treatment and sample carrier plate (1200) can be utilised for different screening interrogations of a sample. The combined sample treatment and sample carrier plate (1200) is placed in a vacuum fixture (1213). The individual array positions (1210) of the combined sample treatment and sample carrier plate are filled with different types of media, e.g. the array positions (1210) on the combined sample treatment and sample carrier plate (1200) can be filled with 96 different antibodies (bound to media). A sample (1212) is then dispensed into the basin (1211) surrounding the combined sample treatment and sample carrier plate (1200) and the sample solution (1212) is after a sufficient incubation time drawn/aspirated through all the positions. The 96 analysis positions are finally analysed individually. This type of screening interrogations of a sample can naturally be conducted in any format, e.g. with 12 individual basins (1211) on the combined sample treatment and sample carrier plate (1200), each basin (1211) covering 4×2 array positions. This would allow for 12 individual samples (1212) to be processed, each on 8 different medium types.

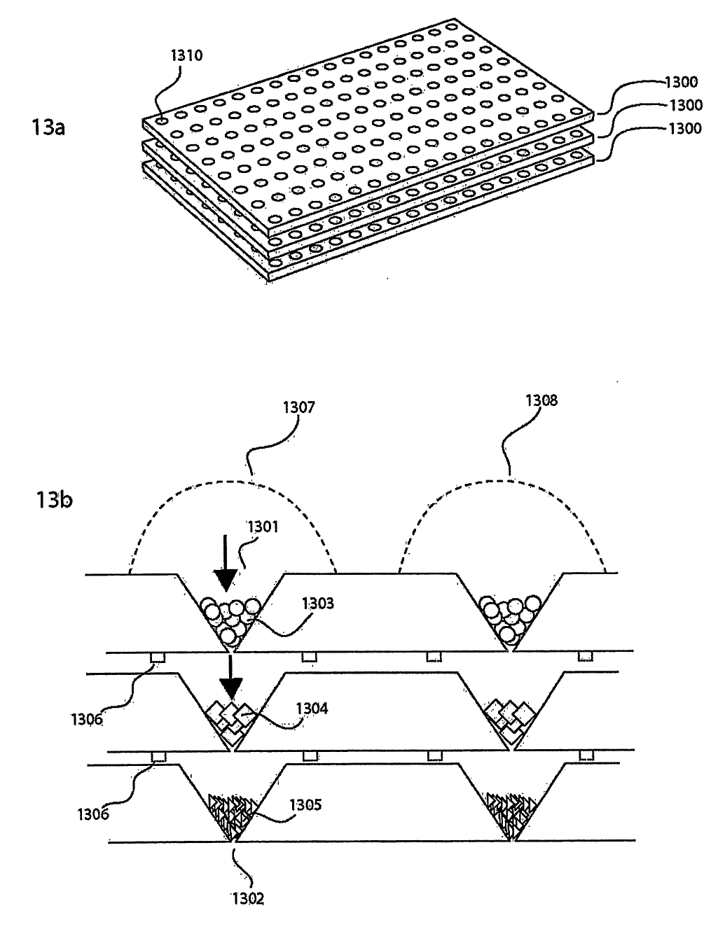

As is shown in FIG. 13a, by stacking of several combined sample treatment and sample carrier plates (1300) with matching array positions (1310) additional sample treatments can be performed, either simultaneously by drawing the sample through the stack of combined sample treatment and sample carrier plates (1300) or consecutively by first doing sample treatment on one combined sample treatment and sample carrier plate (1300) and then put that one on top of another combined sample treatment and sample carrier plate (1300) and displace the analytes to that combined sample treatment and sample carrier plate (1300). Thus stacking of combined sample treatment and sample carrier plates (1300) can be performed in any number or order to facilitate any known sample treatment process.

FIG. 13b shows an example of stacking three combined sample treatment and sample carrier plates. The two samples (1307, 1308) at the inlets (1301) can be processed by drawing/aspirating them through the three different medium types, e.g. a hydrophilic medium (1303), a hydrophobic medium (1304) and a strong-cation exchange medium (1305), of the stack of the combined sample treatment and sample carrier plates by applying a vacuum to the outlets of the combined sample treatment and sample carrier plate at the bottom. The stack is then disassembled and each combined sample treatment and sample carrier plate is eluted and analysed separately before collecting the data for each sample.

Another possible example is that the samples (1307, 1308) first are processed on one combined sample treatment and sample carrier plate where the medium (1303) has a selective antibody. This combined sample treatment and sample carrier plate is then placed/stacked over a combined sample treatment and sample carrier plate with an IMAC medium (1304) and the selectively captured analyte are eluted from the antibody medium (1303) and captured on the IMAC medium (1304). The combined sample treatment and sample carrier plate with the antibody medium (1303) is then removed and the analyte on the IMAC medium (1304) is subjected to a sample treatment process, e.g. enzymatic digestion, while captured on the medium (1304) and then placed over a combined sample treatment and sample carrier plate with a hydrophobic medium (1305). The resulting analytes on the IMAC medium (1304) are eluted and captured on the hydrophobic medium (1305). The analytes are then subjected to a sample treatment, e.g. washing, and eluted onto the analysis zone surrounding the outlet (1302) and this combined sample treatment and sample carrier plate is analysed with LDI MS.

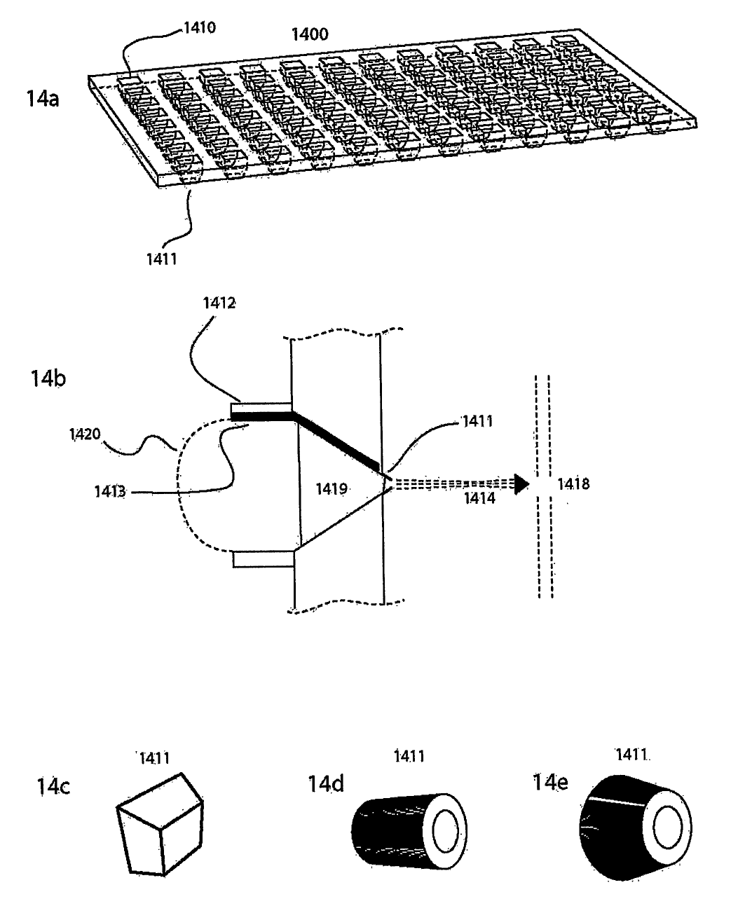

FIG. 14a shows an alternative embodiment of the combined sample treatment and sample carrier plate (1400) having multiple array positions (1410). The outlet (1411) is a nozzle that allows for generation of an electrospray or nanospray that can be delivered to an ESI MS instrument.

FIG. 14b shows a side view of one array position (1410) having an inlet (1420) that can hold a volume of liquid due to the structuring (1412). The inlet (1420) and the outlet/nozzle (1411) define a compartment that can be loaded with medium (1419). The surface of the compartment has an electrode (1413) for applying the voltage necessary for electrospray/nanospray (1414) generation. The electrospray/nanospray (1414) is delivered to an ESI MS instrument by aligning the array positions (1410) on the combined sample treatment and sample carrier plate (1400) that is to be addressed to the inlet of the ESI MS instrument (1418).

FIG. 14c-e shows some possible nozzle (1411) embodiments, FIG. 14c a pyramidal nozzle, FIG. 14d a cylindrical nozzle and FIG. 14e a conical nozzle.

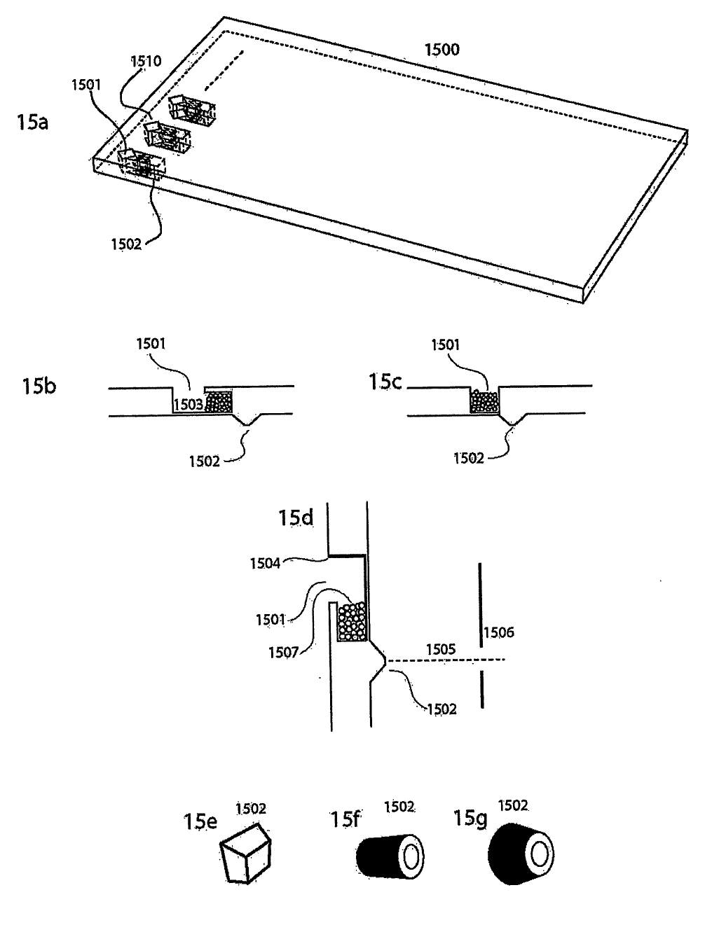

FIG. 15a shows another alternative embodiment of the combined sample treatment and sample carrier plate (1500) having multiple array positions (1510), the outlet (1502) is a nozzle that also allows for generation of an electrospray or nanospray that can be delivered to an ESI MS instrument.

FIG. 15b shows a side view of an array position (1510) embodiment. The array position has an inlet (1501), an outlet (1502) and a compartment (1503) that can be filled with medium. In this embodiment the inlet and outlet does not define the volume of the compartment. Instead the compartment extends horizontally in the material comprising combined sample treatment and sample carrier plate. This allows for a compartment of a larger volume, and thus higher capacity, as compared to a compartment defined solely by the inlet and outlet, going through the combined sample treatment and sample carrier plate.

FIG. 15c is another embodiment of the array position (1510) design depicted in FIG. 15b, but here with an array position with an inlet (1501) and outlet (1502) that defines the compartment volume.

FIG. 15d shows a side view of one array position (1510) having an inlet (1420) that can be filled with liquid. Due to the dimensions and geometry it is possible to fill the array position with liquid from inlet (1501) to outlet/nozzle (1502) by capillary force. The surface of the compartment (1503) has an electrode (1504) for applying the voltage necessary for electrospray/nanospray (1505) generation. The electrospray/nanospray (1505) is delivered to a ESI MS instrument by aligning the array positions (1510) on the combined sample treatment and sample carrier plate (1500) that is to be addressed to the inlet of the ESI MS instrument (1507).

FIG. 15e-g shows some possible nozzle (1502) embodiments, FIG. 15e a pyramidal nozzle, FIG. 15f cylindrical nozzle and FIG. 15g conical nozzle.

Example of a Use Cycle with the Combined Sample Treatment and Sample Carrier Plate During Solid-Phase Extraction of Peptides

1. A defined volume/amount of medium (beads) is added into each well of a 96-well microtitreplate containing different sample solutions in each well.
2. CAPTURE—An appropriate incubation time is allowed for the desired biomolecules to bind to the medium passes.
3. The combined sample treatment and sample carrier plate is placed in its vacuum fixture with the inlets facing up and an appropriate vacuum is applied (under the combined sample treatment and sample carrier plate)
4. LOADING—The sample solution containing the medium is aspirated from a well position of the 96-well microtitreplate by pipetting and transferred to the corresponding position on the combined sample treatment and sample carrier plate. The vacuum makes the sample solution pass through the outlet of the addressed position, while the medium (with the desired biomolecules captured) becomes trapped in the medium compartment of that position. To ensure that all the medium from the position of the 96-well microtitreplate is transferred an additional volume of liquid (preferably wash solution) is added to the well, aspirated (together with any remaining medium) and transferred to the currently addressed position of the combined sample treatment and sample carrier plate. This process is repeated for all the 96 sample positions.
5. WASH—To each position of the combined sample treatment and sample carrier plate, now containing medium, an appropriate volume of washing solution is added and drawn through by the vacuum applied. This may be repeated until one is sure that only the desired biomolecules are left bound to the medium.
6. If a particularly dirty sample has been processed the vacuum is turned off and any undesired contaminations that have ended up on the analysis positions on the side of the outlets is washed away.
7. ELUTION—Again an appropriate vacuum is applied and (typically lower then during loading and washing) to each of the positions a defined elution fluid, causing the captured biomolecules to let go from the medium, is added. The biomolecules are now transferred to the analysis zone (around the outlet).
8. LDI ANALYSIS—The combined sample treatment and sample carrier plate is turned upside down (outlets facing upwards) and placed in a substrate that allows insertion in the LDI instrument. The individual positions are analysed and data is recorded from each position.

### CONCLUSION

The present invention provides a combined sample treatment and sample carrier plate for analysis with LDI (laser desorption ionization). The sample treatment and carrier plate is a flow-through array device where each individual array position has an inlet hole for loading, a compartment of a defined volume and an outlet hole or several outlet holes. The compartment is used to trap a medium (beads, particles, membrane or Empore disc pieces), for affinity selection of biomolecules, preferably beads with a dimension chosen in away that ensures entrapment of the beads in the compartment. The compartment can also be filled with the medium by in-situ polymerisation. The surface surrounding the outlet hole/holes serves as the sample analysis position.

The inlet hole may also define the volume of the compartment for retaining the medium. The inlet may extend vertically in the substrate comprising the combined sample treatment and sample carrier plate and may be of any geometrical shape or cross-section, e.g. cylindrical, square, rhombic, conical or pyramidal. The bottom of this inlet/compartment may have one outlet per inlet or several outlets per inlet position. The outlets may also be of any geometrical shape.

The inlet hole may alternatively lead to a channel, defining the medium compartment, that extends horizontally in the substrate comprising the combined sample treatment and sample carrier plate. This channel may be of any geometrical shape, e.g. cylindrical, square, rhombic, conical or pyramidal. The channel contains a restriction (such as a heel, grid) that retains the medium and has an outlet.

The target plate is preferably designed in such a way that it can be adhered to an ordinary LDI (MALDI, SELDI, DIOS) target by any known means, but preferably a support that accommodates the combined sample treatment and sample carrier plate by physical means. Alternatively the combined sample treatment and sample carrier plate is designed in such a way that it can be fed directly into the MS instrument (without any support).

The combined sample treatment and sample carrier plate can be manufactured by any known means of fabrication, such as microfabrication in silicon, metal, or a polymeric material.

The present invention also relates to use of the sample treatment and carrier device for mass spectrometric analysis. The analyte that is processed may be a biomolecule (peptide, protein, oligonucelotide) or an organic molecule suitable for MS analysis on the presented sample treatment and carrier plate.

The polymeric material may be plated with metal e.g., Au, Ag, Cu, Pt, in order to provide a conductive material permitting ionization of the analyte biomolecules.

The design may be used to trap medium, preferably beads, to allow selective capture of a biomolecule. The trapped medium can be of any know functionality, such as RP, SCX, IEX. The captured entity on the medium can also be a biomolecule, such as an antibody, peptide, DNA, protein, carbohydrate and the analyte is eluted onto the analysis zone. The medium loaded can be of a singular type, or layered by type or a mixture of media having different affinity properties.

The solutions and/or media are delivered to the device by any known means, such as piezodispensing, pipetting or any other form of dispensing

The bead/medium, sample solution, wash solution, other solutions (e.g. containing reactants) are loaded and drawn through by applying a vacuum to one side of the plate, preferably the side with the analysis zone. Alternatively, the solutions are passed through the device by applying capillary force or pressure.

The plate may be mated to a cover plate that allows elution by pressure e.g. by insertion of a pipette tip into/above the inlet.

The analyte solution containing the target biomolecules may be passed through the medium and bound to this for processing and subsequent elution onto the sample analysis position for analysis, preferably with mass spectrometry, preferably LDI MS (MALDI, SELDI, DIOS). The sample may be bound to the medium in an external container, such as a microtitreplate, Eppendorf tube or similar, and the medium is then transferred to the device for subsequent treatment and analysis.

The sample may be bound by passing the analyte solution through the medium in the compartment and subjected to treatment (chemical or enzymatic) before elution onto the sample analysis position. Alternatively the eluted analyte biomolecules is subjected to an enzymatic or chemical reaction on the analysis zone/position and then analysed with MS. The reagent may be supplied to the position by any means e.g. pipetting, dispensing or contact printing. The analyte may be subjected to an enzymatic or chemical sequencing step either while still bound to the medium or on the analysis zone prior to the MS analysis

One or more additional plate/plates may be placed above or below for performing sample treatment, such as digestion of proteins captured on the medium. The additional plate can be a replicate of the first or the additional plate can be any other design and material. Two or several of these plates may be stacked together with different media in each plate and the analyte solution is passed through the stacked plates for subsequent analysis of each individual plate (plate 1 IMAC/plate 2 RP, plate −SH/plate 2 RP . . . )

The different compartments on the combined sample treatment and sample carrier plate may be filled with media having different capture moieties and a sample solution passed through these compartments in order to selectively capture specific biomolecules on the different medium.

The bound analyte may be eluted onto the sample analysis position for analysis with a complementary technique, such as fluorescence or SPR prior to MS analysis.

The present device may be subjected to a surface treatment to promote, inhibit or otherwise alter binding, loading or elution.

The sample analysis zone may be surface-modified to provide a specific behaviour, such as providing a small sample spot, enhanced crystallisation or enabling direct ionisation from the surface (DIOS).

The analysis zone may be defined by adding a structural support.

The analysis zone may be defined by patterning of hydrophobic/hydrophilic layers.

The analysis zone may be altered in any known means to promote crystallisation of the eluted analytes/matrix such as porous silicon.

The device may be used for differential expression analysis. E.g. samples from different conditions are mixed and the resulting analytes or some specific resulting analytes are captured and analysed by MS. In order to make the differential expression of the analytes visible in the MS analysis any known type of isotopic labeling method can be applied. The medium used for capture may be of any type (RP, biotin, −SH).

The analyte biomolecules while still captured on the medium or after elution onto the analysis zone of the combined sample treatment and sample carrier plate may be analysed by optical means, such as fluorescence scanner, microscopy, scintillation detection or similar.

The eluted analytes may be analysed by (LDI) MS to deduce M_{W}, perform peptide map fingerprinting, or MS/MS in order to establish the sequence and/or the identity and/or the quantitative level of the analyte. The analyte is eluted onto the analysis zone and analysed by a LDI MS/MS technique, such as MALDI TOF-TOF or MALDI Q-TOF

The medium is used to capture specifically modified analyte. E.g IMAC can be used and the captured analyte (phosphopeptide/s) is analysed (identified) by MS/MS.

In another application the phosphopeptide can be subjected to a dephosphorylation step while captured to the medium, or on the analysis zone in order to confirm phosphorylation of that peptide.

The analytes bound to the medium may be subjected to a chemical reaction to enhance MS detection or promote/direct fragmentation of the analyte during MSⁿ analysis either while still bound to the medium or on the analysis zone, e.g. conversion of lysine to homoarginine or trimetylation.

The analytes may be displaced from the medium with a solution that also contains the matrix necessary for LDI (eg, DHB,CHCA,FA,SA,THAP, . . . ), said matrix crystallising on the analysis zone.

The analytes may be displaced from the medium using only elution solution and the matrix necessary for LDI is added to the elution zone (analysis zone) after by any known means such as pipetting, dispensing, e-spray.

The device may be used in a pressure, humidity and/or temperature controlled environment to enable loading, elution, biological, chemical or other features.

The combined sample treatment and sample carrier plate may be used to collect fractions from a separation (e.g. liquid chromatography, isoelectric focusing, capillary electrochromatography) of biomolecules for subsequent sample treatment of the captured biomolecules and finally analysis.

The device may be used to store samples bound to the medium for later analysis.

The scope of the invention is only limited by the claims below.

1. A device for combined sample treatment and sample carrying, comprising a plate with inlets at one side connected to respective compartments situated at respective array positions for receiving samples to be treated and analysed, wherein each compartment is in communication with an outlet enabling fluid flow through the compartment.

2. A device according to claim 1, wherein the respective outlet simultaneously serves as a restriction for retaining a medium.

3. A device according to claim 1, wherein the respective outlet comprises a restriction for retaining a medium.

4. A device according to claim 3, wherein the outlet comprises a structure with restriction apertures.

5. A device according to claim 3, wherein the outlet comprises a permeable membrane.

6. A device according to claim 1, wherein the outlet is arranged at the other side of the plate opposite the inlet, wherein the respective compartment is formed between the inlet and the outlet.

7. (canceled)

8. A device according to claim 1, wherein the outlet is arranged at the other side of the plate displaced from the inlet, wherein the respective compartment is formed between the inlet and the outlet.

9. A device according to claim 1, wherein the outlet is arranged at the same side of the plate displaced from the inlet, wherein the respective compartment is formed between the inlet and the outlet.

10. A device according to claim 8 wherein the respective compartment is formed as a channel directed in the same plane as the plate.

11. A device according to claim 10, wherein the respective compartment comprises a restriction for retaining a medium.

12. A device according to claim 11, wherein the restriction comprises a grid.

13. (canceled)

14. (canceled)

15. A device according to claim 1, wherein the respective inlet comprises a structure, such as bars, for hindering matter to enter the compartment.

16. A device according to claim 1, wherein an analysis zone is arranged at each outlet.

17. A device according to claim 16, wherein the analysis zone is structured to achieve a well defined analysis area.

18. (canceled)

19. A device according to claim 17, wherein the analysis zone structure comprises a patterned structure, such as a hydrophilic layer or a hydrophobic layer, or a nanoporous surface or a planar surface or a combination thereof.

20. A device according to claim 1, wherein a structured zone is arranged at each inlet.

21. (canceled)

22. A device according to claim 20, wherein the structured zone comprises a patterned structure, such as a hydrophilic layer or a hydrophobic layer, or a nanoporous surface or a planar surface or a combination thereof.

23. A device according to claim 1, wherein one or both of the sides of the device is made hydrophobic.

24. (canceled)

25. (canceled)

26. (canceled)

27. A device according to claim 1, wherein the device is arranged to generate electrospray.

28. A device according to claim 27, wherein the device comprises a nozzle at each outlet, and an electrode at each compartment.

29. A device according to claim 28, wherein the nozzle is in the form of a pyramidal nozzle, a cylindrical nozzle or a conical nozzle.

30. (canceled)

31. (canceled)

32. (canceled)

33. A method for analysis of samples using a combined sample treatment and sample carrier device comprising a plate with inlets at one side connected to respective compartments situated at respective array positions for receiving samples to be treated and analysed, each compartment being in communication with an outlet enabling fluid flow through the compartment comprising the steps of:
supplying an external container with a sample;
optionally, subjecting the sample to a first treatment in the external container;
transferring the sample to the combined sample treatment and sample carrier device;
subjecting the sample to a second treatment exploiting fluid flow through the device, wherein a medium is trapped in the device.

34. (canceled)

35. (canceled)

36. (canceled)

37. A method according to claim 33, wherein the second treatment comprises washing, wherein a washing solution is drawn through the combined sample treatment and sample carrier device.

38. A method according to claim 33, wherein the second treatment comprises elution, wherein an elution solution is drawn through the combined sample treatment and sample carrier device.

39. A method according to claim 33, wherein the second treatment comprises transferring the sample to an analysis zone on the combined sample treatment and sample carrier device.

40. A method according to claim 33, wherein the sample is drawn through the combined sample treatment and sample carrier device to the analysis zone.

41. A method according to claim 39 wherein the sample is subjected to crystallisation in the analysis zone.

42. A method according to claim 41, wherein the sample is drawn through the combined sample treatment and sample carrier device to the analysis zone with a solution containing a matrix for LDI, such as DHB, CHCA, FA, SA and THAP.

43. A method according to claim 39, wherein the analysis zone is situated on the underside, and the combined sample treatment and sample carrier device is turned upside down for subjecting said plate to an analysis instrument.

44. A method according to claim 33, wherein the second treatment comprises electrospraying the sample to an analysis instrument.

45. A method according to claim 33, wherein the combined sample treatment and sample carrier device is placed in a suction fixture that is operated to aspirate fluid through the device as and when required in the respective steps.

46. A method according to claim 34, wherein the medium has a selective affinity for various biomolecules.

47. A method according to claim 46, wherein the medium has hydrophilic, hydrophobic, cation exchange, RP, SCX, IMAC or IEX functionality.

48. A method according to claim 46 wherein the medium comprises beads, particles, membranes or Empore disc pieces.

49. A method according to claim 46, wherein the medium is supplied to the combined sample treatment and carrier plate by in-situ (in-chip) polymerisation, such as a medium of porous polymer monolith.

50. A method according to claim 33, wherein the combined sample treatment and sample carrier device comprises several stacked plates.

51. A method according to claim 50, wherein different media are carried by the different plates.

52. A method according to claim 51, wherein the second treatment is performed simultaneously in the stacked plates.

53. A method according to claim 51, wherein that part of the second treatment is performed simultaneously in the stacked plates, and then the stack is disassembled for separate treatment of the plates.

54. A method according to claim 33, wherein the combined sample treatment and sample carrier device after the second treatment is subjected to analysis.

55. A method according to claim 54, wherein the analysis is optical, such as fluorescence detection, laser detection, scintillation detection or microscopy.

56. A method according to claim 54, wherein the analysis comprises mass spectroscopy (MS), such as LDI, ESI, SELDI, DIOS, MALDI TOF-TOF, MALDI Q-TOF.

57. A method according to claim 33, wherein the second treatment comprises treatment with reagents for enzymatic or chemical reactions.

58. (canceled)

59. (canceled)

60. (canceled)

61. (canceled)

62. (canceled)

63. A device according to claim 9, wherein the respective compartment is formed as a channel directed in the same plane as the plate.

64. A device according to claim 63, wherein the respective compartment comprises a restriction for retaining a medium.

65. A device according to claim 64, wherein the restriction comprises a grid.

66. A method according to claim 40, wherein the sample is subjected to crystallisation in the analysis zone.

67. A method according to claim 66, wherein the sample is drawn through the combined sample treatment and sample carrier device to the analysis zone with a solution containing a matrix for LDI, such as DHB, CHCA, FA, SA and THAP

68. A method according to claim 40, wherein the analysis zone is situated on the underside, and the combined sample treatment and sample carrier device is turned upside down for subjecting said plate to an analysis instrument.
